(19) United States
(12) Patent Application Publication
(10) Pub. No.: US 2012/0157467 A1
(43) Pub. Date: Jun. 21, 2012

(54) COMPOSITION AND USE FOR PHOSPHODIESTERASE INHIBITORS

(76) Inventor: Alex Chervinsky, Brooklyn, NY (US)

(21) Appl. No.: 12/971,229

(22) Filed: Dec. 17, 2010

(51) Int. Cl. A61K 31/497; A61K 31/522; A61P 17/00; A61K 31/519

(52) U.S. Cl. 514/252.16; 514/262.1; 514/263.34

(57) ABSTRACT

Compositions and methods for enhancing the aesthetic appearance of the lip or lips of a human achieved by applying to the surface of the lip a composition containing a phosphodiesterase inhibitor, preferably a pyrazolopyrimidinone such as sildenafil or its citrate salt.

### FIELD OF THE INVENTION

The present invention relates generally to a composition and method for lip enhancement or lip augmentation, and particularly to a composition and method for enhancing the appearance of the lips without use of an injection or plastic surgery. More particularly, the subject invention relates to the use of a composition comprising a phosphodiesterase (PDE) inhibitor, preferably a cyclic adenosine monophosphate phosphodiesterase (c-AMP PDE) inhibitor or a cyclic guanosine monophosphate phosphodiesterase (c-GMP PDE) inhibitor, such as a pyrazolopyrimidinone, and preferably sildenafil and it salts, for enhancing the aesthetic appearance, e.g., the size, shape, or color, of the lips.

### BACKGROUND OF THE INVENTION

Active ingredients of the subject invention are not heretofore described for use as a lip enhancement. Pyrazolopyrimidinones are known potent and selective inhibitors of cyclic guanosine 3′,5′-monophosphate phosphodiesterase (cGMP PDE). As a consequence of this selective PDE inhibition, cGMP levels are elevated, which in turn can give rise to beneficial platelet anti-aggregatory, anti-vasopastic and vasodilatory activity, as well as potentiation of the effects of endothelium-derived relaxing factor (EDRF) and nitrovasodilators. Thus, pyrazolopyrimidinones are known to have utility in the treatment of a number of disorders, including stable, unstable and variant (Prinzmetal) angina, hypertension, congestive heart failure, atherosclerosis, conditions of reduced blood vessel patency e.g. post-percutaneous transluminal coronary angioplasty (post-PTCA), peripheral vascular disease, stroke, bronchitis, chronic asthma, allergic asthma, allergic rhinitis, glaucoma, and diseases characterized by disorders of gut motility, e.g. irritable bowel syndrome (IBS). The pyrazolopyrimidinones include sildenafil and its citrate salt, which is the active component in the erectile dysfunction (ED) treatment sold as VIAGRA®.

Other cGMP PDE inhibitors are also marketed for the treatment of ED. For example, the beta-carboline compounds, (6R,12aR)-2,3,6,7,12,12a-hexahydro-2-methyl-6-(3,4-methylenedioxyphenyl)pyrazino[2′,1′:6,1]pyrido[3,4-b]indole-1,4-dione; and (3S,6R,12aR)-2,3,6,7,12,12a-hexahydro-2,3-dimethyl-6-(3, 4-methylenedioxyphenyl)pyrazino[2′,1′:6,1]pyrido[3,4-b]indole-1,4-dione, and the salts and solvates thereof, which include the compound known as tadalafil, which is marketed as CIALIS®. Beta-carboline compounds, and pharmaceutical compositions containing the beta-carbolines are described in U.S. Pat. No. 5,859,006. International PCT publication WO 97/03675 discloses use of beta-carboline compounds in the treatment of sexual dysfunction.

Compounds in the class, 2-phenyl-substituted imidazotriazinones, are disclosed in DE2811780 and are described as bronchodilators having spasmolytic activity and inhibitory activity against phosphodiesterases which metabolize cyclic adenosine monophosphate (cAMP-PDEs). The imidazotriazinones include vardenafil, and its hydrochloride salt, marketed as LEVITRA® for ED.

The methylated xanthine derivatives, such as aminophylline and theophylline are commonly used in the treatment of bronchial asthma. These compounds are competitive nonselective phosphodiesterase inhibitors, which raise intracellular cAMP, activate PKA, inhibit TNF-alpha and leukotriene synthesis, and reduces inflammation and innate immunity and are concomitantly nonselective adenosine receptor antagonists. Aminophylline is known to be less potent and shorter-acting than theophylline.

In recent years it has become fashionable for people, particularly women, to have fuller looking lips. The most common way in which fuller looking lips are attained is through a lip augmentation procedure, and often through the injection of fat or collagen (typically non-human collagen, such as bovine or porcine collagen) into the lip or by surgical implant or graft of a compatible material.

Injected collagen may last for over a year, but in some individuals will only last for several months because it is eventually digested by the body. The known risks associated with collagen replacement therapy can be classified into two categories: those associated with the collagen material itself, and those associated with the injection procedure. The primary known risk of the collagen material is an allergic reaction, which can result in prolonged redness, swelling, firmness and itching or, in rare instances, in formation of an abscess (cyst) that may result in hardness or a scar. A primary risk from the injection procedure is bruising and swelling at the injection site or, in rare cases, necrosis (tissue sloughing, or shedding, and resulting scab or scar formation) at the injection site.

For a more permanent lip augmentation, material such as Gore-tex strands or Alloderm may be surgically implanted directly into the lip. Alloderm is highly processed human skin (dermis) that can be grafted or implanted into the upper or lower lip to give your lips a fuller appearance.

While these risks do not manifest themselves in every patient, many prospective patients choose to not undergo lip augmentation procedures because of the fear of these risks. Because of these fears, these risks, and the time involved with undergoing lip augmentation procedures, there exists a need for a faster, easier, and less dangerous way of augmenting or enhancing lips.

### SUMMARY OF THE INVENTION

A composition and method in accordance with the subject invention can address the problems and risks associated with injectable or surgical lip enhancement procedures. Preferably, the invention comprises a composition comprising an effective amount of at least one phosphodiesterase (PDE) inhibitor and, more preferably, at least one c-AMP or c-GMP PD inhibitor such as sildenafil, tadalafil, vardenafil, or a methylated xanthine or salts thereof, which can be topically applied to the lips for aesthetic or cosmetic lip enhancement or augmentation.

The novel use and method comprises application, preferably topical application to the outer surface of the lip, of a composition comprising about 0.5% to about 20%, and preferably about 1% to about 10%, of at least one PDE inhibitor to achieve enhancement or augmentation of the appearance of the lip or lips. The composition can comprise additional ingredients, such as one or more additional active ingredients, including one or more additional PDE inhibitors, one or more solvents, emollients, fillers, waxes, oils, pharmaceutically or cosmetically acceptable excipients, and the like, as conventionally used and known in the art. The composition of the subject invention can further comprise additional medicaments or active ingredients used in medicated lip balms or cosmetic lipsticks. Accordingly, the composition can be used as a topical or transdermal lip enhancer and can further be used as a medicament or a cosmetic.

In a preferred embodiment, the composition comprises about 1% sildenafil citrate, about 6% aminophylline anhydrous, and about 12% L-arginine, the balance of the composition comprising pharmaceutically or cosmetically acceptable excipients. In an alternative embodiment, the composition can comprise about 1% sildenafil citrate, about 6% naltrexone HCl, and about 12% L-arginine.

### DETAILED DESCRIPTION

The subject invention concerns a composition which can be topically applied to the outer surface of the lip or lips for enhancing or augmenting the aesthetic appearance of the lip or lips of an animal, preferably a human. The subject invention further comprises a novel method of use for a phosphodiesterase (PDE) inhibitor, wherein the method comprises providing a composition comprising at least one PDE inhibitor and applying the composition to the outer surface of the lip or lips to enhance the appearance (e.g., increase the size or fullness) of the lip or lips.

In a preferred embodiment, the subject composition comprises at least one c-AMP or c-GMP PDE inhibitor. The c-AMP and c-GMP PDE inhibitors include the pyrazolopyrimidinones, beta-carbolines, imidazotriazinones, methylated xanthines, and derivatives, isomers, hydrates, and salts thereof.

Preferred pharmaceutically acceptable salts of these compounds include the hydrochloride (HCl), hydrobromide (HBr), sulphate or bisulphate, phosphate or hydrogen phosphate, acetate, citrate, fumarate, gluconate, lactate, maleate, succinate and tartrate salts, or pharmaceutically acceptable metal salts, particularly alkali metal salts, such as the sodium and potassium salts.

Preferred pyrazolopyrimidinones can include the compounds:
5-[2-allyloxy-5-(4-methylpiperazinylsulphonyl)phenyl]-1-methyl-3-n-propyl-1,6-dihydro-7H-pyrazolo[4,3-d]pyrimidin-7-one;
5-[2-ethoxy-5-(piperazinylsulphonyl)phenyl]-1-methyl-3-n-propyl-1,6-dihydro-7H-pyrazolo[4,3-d]pyrimidin-7-one;
5-[2-ethoxy-5-(4-methylpiperazinylsulphonyl)phenyl]-1-methyl-3-n-propyl-1,6-dihydro-7H-pyrazolo[4,3-d]pyrimidin-7-one;
5-{2-ethoxy-5-[4-(2-propyl)piperazinylsulphonyl]phenyl}-1-methyl-3-n-propyl-1,6-dihydro-7H-pyrazolo[4,3-d]pyrimidin-7-one;
5-{2-ethoxy-5-[4-(2-hydroxyethyl)piperazinylsulphonyl]phenyl}-1-methyl-3-n-propyl-1,6-dihydro-7H-pyrazolo[4,3-d]pyrimidin-7-one;
1-methyl-5-[5-piperazinylsulphonyl)-2-n-propoxyphenyl]-3-n-propyl-1,6-dihydro-7H-pyrazolo[4,3-d]pyrimidin-7-one; and
5-{5-[4-(2-hydroxyethyl)piperazinylsulphonyl]-2-n-propoxyphenyl}-1-methyl-3-n-propyl-1,6-dihydro-7H-pyrazolo[4,3-d]pyrimidin-7-one.

A preferred pyrazolopyrimidinone compound used in a composition and method according to the subject invention is sildenafil, and more preferably, sildenafil citrate.

Preferred beta-carboline compounds include:
Cis-2,3,6,7,12,12a-hexahydro-2-(4-pyridylmethyl)-6-(3,4-methylenedioxyphenyl)-pyrazino[2′,1′:6,1]pyrido[3,4-b]indole-1,4-dione;
Cis-2,3,6,7,12,12a-hexahydro-6-(2,3-dihydrobenzo[b]furan-5-yl)-2-methyl-pyrazino[2′,1′:6,1]pyrido[3,4-b]indole-1,4-dione;
Cis-2,3,6,7,12,12a-hexahydro-6-(5-bromo-2-thienyl)-2-methyl-pyrazino[2′,1′:6,1]pyrido[3,4-b]indole-1,4-dione;
Cis-2,3,6,7,12,12a-hexahydro-2-butyl-6-(4-methylphenyl)-pyrazino[2′,1′:6,1]pyrido[3,4-b]indole-1,4-dione;
(6R,12aR)-2,3,6,7,12,12a-Hexahydro-2-isopropyl-6(3,4-methylenedioxyphenyl)-pyrazino[2′,1′:6,1]pyrido[3,4-b]indole-1,4-dione;
(6R,12aR)-2,3,6,7,12,12a-Hexahydro-2-cyclopentyl-6-(3,4-methylenedioxyphenyl)-pyrazino[2′,1′:6,1]pyrido[3,4-b]indole-1,4-dione;
(6R,12aR)-2,3,6,7,12,12a-Hexahydro-2-cyclopropylmethyl-6-(4-methoxyphenyl)-pyrazino[2′,1′:6,1]pyrido[3,4-b]indole-1,4-dione;
(6R,12aR)-2,3,6,7,12,12a-Hexahydro-6-(3-chloro-4-methoxyphenyl)-2-methyl-pyrazino[2′,1′:6,1]pyrido[3,4-b]indole-1,4-dione;
(6R,12aR)-2,3,6,7,12,12a-Hexahydro-2-methyl-6-(3,4-methylenedioxyphenyl)-pyrazino[2′,1′:6,1]pyrido[3,4-b]indole-1,4-dione;
(6R,12aR)-2,3,6,7,12,12a-Hexahydro-6-(3,4-methylenedioxyphenyl)-pyrazino[2′,1′:6,1]pyrido[3,4-b]indole-1,4-dione;
(5aR,12R,14aS)-1,2,3,5,6,11,12,14a-Octahydro-12-(3,4-methylenedioxyphenyl)-pyrrolo[1″,2″:4′,5]pyrazino[2′,1′:6,1]pyrido[3,4-b]indole-5-1,4-dione; and
(3S,6R,12aR)-2,3,6,7,12,12a-hexahydro-2,3-dimethyl-6-(3,4-methylenedioxyphenyl)pyrazino[2′,1′:6,1]pyrido[3,4-b]indole-1,4-dione.

A preferred beta-carboline compound used in a composition and method of the subject invention is tadalafil, and can include salts of tadalafil.

Preferred imidazotriazinones are disclosed in U.S. Pat. No. 6,362,178, which is incorporated herein by reference, in its entirety. One preferred imidazotriazinone compound used in a composition and method according to the subject invention is vardenafil, most preferred in its hydrochloride salt form.

A composition of the subject invention can further comprise an amino acid such as L-arginine. L-arginine is useful to produce nitric oxide, a compound in the body that relaxes blood vessels. This vasodilating property of amino acids is known to be useful in treating conditions such as atherosclerosis, erectile dysfunction, and intermittent claudication.

A composition of the subject invention can further comprise a methylated xanthine compound, e.g., aminophylline or theophylline, which is a known bronchodilator and PDE inhibitor. Preferably, a composition and method of the subject invention comprises aminophylline anhydrous.

Alternatively, the composition can further comprise an opioid receptor antagonist, such as naltrexone, or its pharmaceutically acceptable salt, e.g., naltrexone HCl, or a derivative of naltrexone, e.g., methylnaltrexone. Other useful opioid receptor antagonists include naloxone, nalorphine, and naloxazone. Naltrexone is the N-cyclopropylmethyl derivative of oxymorphone.

Compositions of the subject invention can be provided as a dry powder, in liquid form as a solution or suspension, or as a cream, gel, lotion, paste, salve, ointment, or balm which can be applied or is spreadable onto the surface of the lip. These compositions comprise active ingredients, preferably in combination with excipients or carriers which form a base in which the active ingredient is mixed, dissolved, or suspended.

In one preferred embodiment, the composition of the subject invention is provided as a lip balm or ointment, and can be in a stick form, such as a lipstick or chapstick form. Many lip balms are commercially available in a stick form, which typically contain a wax, in amounts of 20% wt/wt or more of the composition. Waxes can be used in the composition to give the composition sufficient structure to maintain a stick form under shipping and normal use conditions. Petroleum derived waxes such as ozokerite, paraffin, and microcrystalline petroleum based waxes are commonly used as they provide a robust stick structure and are relatively inexpensive materials.

Among the waxes which may be used in accordance with the subject invention are mineral waxes, such as microcrystalline waxes, paraffin, petrolatum, petroleum jelly, ozokerite and montan wax; animal waxes such as beeswax, lanolin and derivatives thereof; plant waxes such as candelilla wax, ouricurry wax, carnauba wax, Japan wax, cocoa butter, cork fibre wax or sugar cane wax; hydrogenated oils, fatty esters and glycerides which are solid at 25° C.; synthetic waxes such as polyethylene waxes and the waxes obtained by Fischer-Tropsch synthesis; silicone waxes; mixtures thereof.

The composition may preferably comprise up to 99% by weight of lipophilic component, which may preferably contain from 8 to 40% by weight of waxes and from 60 to 92% by weight of oils. Among the oils which may be envisaged, alone or as a mixture, are mineral oils such as paraffin oil or liquid petroleum jelly; animal oils such as perhydrosqualene or arara oil; plant oils such as sweet almond oil, calophyllum oil, palm oil, castor oil, avocado oil, jojoba oil, olive oil or cereal germ oil; silicone oils such as polydimethyl-siloxane; esters of lanolic acid, of oleic acid, of lauric acid, of stearic acid or of myristic acid, for example; alcohols such as oleyl alcohol, linoleyl alcohol, linolenyl alcohol, isostearyl alcohol or octyldodecanol; alcohol or polyalcohol acetylgly-cerides, octanoates, decanoates or ricinoleates; volatile oils such as cyclotetradimethylsiloxane, cyclopentadimethylsiloxane, cyclohexadimethylsiloxane and methylhexyldimethylsiloxane, or isoparaffins.

Many users of lip balm products prefer “natural” ingredients. Accordingly, use of “natural” waxes such as beeswax or plant derived waxes such as candelilla or carnauba have in some instances been used instead of or as a partial replacement for petroleum wax. Waxes can also provide the benefit of forming a protective layer when applied to the lips. Because conventional petroleum-derived base waxes, though protective, may not replenish lipids and thus may not maintain natural moisture balance and prevent drying, chapping and cracking of lips. Optionally, the composition may contain one or more antioxidants, sunscreens, or preservatives.

Moisturizing ingredients, e.g., oils or “butters” may also be used in a composition of the subject invention. Generally butters contribute skin-conditioning properties to the composition. The composition can comprise one or more botanical butters. A botanical butter includes both pure extracts from a plant fruit or seed and/or extract from a plant fruit or seed combined with additional lipid material to achieve the melting point characteristic and/or lubricity. Preferably the lipid material is derived from a botanical source.

Suitable butters include but are not limited to mango seed butter, raspberry butter, avocado butter, shea butter, olive butter, kuku butter, monoi butter, peach butter, pistachio butter, coconut butter, cocoa butter, pomegranate butter, rose hip butter, sunflower butter, wheat germ butter, apricot butter, babassu butter, cupuacu butter, kokum butter, hazelnut butter, jojoba butter, sesame butter, soy butter, almond butter, meadowfoam seed butter, black current seed butter, cranberry butter and combinations thereof.

Depending on the total number of butters used the total amount of butter is typically about 2% to about 20%. A plurality of butters is desirable as, in addition to emollient and moisturizing properties, these naturally derived substances have a variety of other attributes depending on the botanical source of the butter. For example, butters may vary in their sensory feel and/or have particular components with desired functionalities such as components that enhance lip barrier function, enhance penetration, or have antioxidant properties or the like. Accordingly, a combination of butters provides for modulation of lip feel (such as, for example, suppleness and/or moistness) of the composition as well as a source of natural ingredients with beneficial attributes (such as, for example, emollient, moisturizer, medicament, and/or antioxidant).

Natural moisturizing lip balm products are available commercially, and are usually sold in tubes or pots because as they can be too soft to form a stick that can withstand storage, shipment and/or application to the lips without significant distortion of shape. Application of such balm products is typically accomplished by the user's fingers.

As used herein, a “butter” or “botanical butter” is a fat and/or oil extract of a plant fruit and/or seed characterized by having emollient properties and a melting point near human body temperature.

The terms “about” or “approximately” mean within an acceptable range for the particular parameter specified as determined by one of ordinary skill in the art, which will depend, in part, on how the value is measured or determined, e.g. the limitations of the measurement system. For example, “about” can mean a range of up to 10% of a given value.

“Percent” or “%” as used herein refers to the percentage by weight of the total composition, unless otherwise specified.

An “emollient” is a cosmetic ingredient which can help maintain the soft, smooth and pliable appearance of skin tissue. Typically, emollients impart sensory properties of smoothness and/or softness when the composition is applied to the lips.

“Conventional wax” or “conventional waxes” include petroleum derived waxes, beeswax, carnauba wax and candelilla wax. These waxes may also be referred to herein as “non-jojoba wax” or “non-jojoba waxes”. Jojoba waxes are distinguishable from conventional waxes in that they are derived from the jojoba plant and they have properties that contribute to structure as well as providing substantial emollient and moisturizing properties not characteristic of conventional waxes.

A “lip balm” is a semisolid composition for application to the lips that has protective and/or moisturizing properties. Lip balms may contain medicaments and/or ingredients that promote lip health, such as sunscreens. Lip balms may also include colorants that may or may not impart the color to the lips. A “stick lip balm” is a lip balm formed into a stick shape that is typically dispensed from a container that permits extension and retraction of the lip balm stick from and into the container.

In an exemplary embodiment of the invention, a lip balm base is provided and an effective amount of one or more PDE inhibitors is combined by homogeneously mixing said PDE inhibitor into the base. The preferred PDE inhibitor is selected from a pyrazolopyrimidinone, a beta-carboline, an imidazotriazinone, and a methylated xanthine. The PDE inhibitor is typically provided in an amount of about 0.5% to about 20% wt/wt of the total composition. Preferably, the PDE inhibitor is provided in an amount of about 5-7%, and more preferably the PDE inhibitor is provided in an amount of about 7%, as about 1% of a pyrazolopyrimidinone, beta-carboline, or imidazotriazinone, and about 6% of a methylated xanthine.

Optionally, an amino acid component can be included and homogeneously mixed into the base in combination with the PDE inhibitor. Typically, the amino acid is provided in an amount of about 5% to about 15% wt/wt of the total composition. Preferably, the amino acid is L-arginine, and is used in an amount of about 12% of the total composition.

To obtain emollient properties and desired structure, a mixture of jojoba esters may be used. In view of commercially available jojoba ester products such as Floraesters supplied by FloraTech Americas, 1151 North Fiesta Blvd., Gilbert Ariz. 85233, it may be convenient to use a combination of jojoba ester products.

The composition can further comprise moisturizing oils. Exemplary oils suitable for use in the composition included, but are not limited to, sunflower oil, coconut oil, castor oil, vegetable oil, corn oil, aloe vera oil, canola oil, soybean oil, jojoba oil, olive oil, babassu oil, avocado oil, apricot oil, meadowfoam seed oil, macadamia seed oil, oat kernel oil, palm seed oil, safflower oil, sandalwood oil, sesame oil, almond oil, wheat germ oil, cranberry oil and combinations thereof. In view of consumer preferences it is preferable to use oil from a botanical source. Oils may be included in the composition in amounts of about 5% to about 65%.

The composition may further comprise one or more antioxidants. Antioxidants may protect the composition from oxidation (e.g. becoming rancid) and/or provide lip conditioning benefits upon application to the lips. In view of consumer preferences for “natural” products natural antioxidants such as tocopherols are preferred. Tocopherols, tocopheryl acetate, some botanical butters and green tea extracts are exemplary antioxidants suitable for use in the composition.

Optionally, the composition may further comprise a flavorant. In view of consumer preferences it is preferable to use a natural flavorant material. Flavorants are typically used in amounts of about 0.1% to 5%. Amounts may vary depending on the potency of the flavorant and matrix in which the flavorant is presented. Flavorants may be presented in a botanical lipid matrix, for example.

Optionally, the composition may further comprise natural medicaments including, but not limited to, menthol, camphor, eucalyptus, salicylic acid, and derivatives of salicylic acid. Typically medicaments would be added in amounts of less than about 3%. Amounts may vary depending on the potency of the medicament and the matrix in which the medicament is presented.

Optionally, colorant that imparts color to the composition may be included in the composition. The colorant should not be of an amount, particle size, and/or presented in a matrix that would permit transfer of colorant that imparts a color to the lips during application. Natural colorants such as, for example, plant extracts, natural minerals, or carmine are preferred.

Optionally, a sensate may be included in the composition. A sensate is a composition that initiates a sensory perception such as heating or cooling, for example, when contacted with the skin and/or lips. Exemplary sensates include, but are not limited to, mint extracts, cinnamon extract, and capsaicin. Preferably sensates are derived from natural sources. However, as sensates typically have high potency and accordingly may yield significant impact at low levels, synthetic sensates are within the scope of this invention.

In addition to the components described above, other components which are usually blended into compounded compositions for cosmetic or aesthetic use, including oils, resins, silicone rubber, moisture retaining agents, antioxidants, surface active agents, preservatives, anti-inflammatories, ultraviolet light adsorbents, drugs such as vitamins and hormones, sequestering agents, viscosity adjusting agents and perfumes, can be blended into the composition of the present invention.

For the oil, various hydrocarbons, higher fatty acids, fats and oils, esters, higher alcohols, waxes, silicone oils and fluorocarbon oils including squalene, liquid paraffin, vaseline, microcrystalline wax, ozokerite, ceresin, myristic acid, palmitic acid, stearic acid, oleic acid, isostearic acid, cetyl alcohol, hexadecyl alcohol, oleyl alcohol, cetyl-2-ethyl hexanoate, 2-ethylhexyl palmitate, 2-octyldodecyl myristate, 2-octyldodecylgum ester, neopentylglycol-2-ethyl hexanoate, isooctyric acid triglyceride, 2-octyldodecyl oleate, isopropyl myristate, isostearic acid triglyceride, coconut oil fatty acid triglyceride, olive oil, avocado oil, bees wax, myristyl myristate, mink oil, lanolin, dimethylpolysiloxane, ring dimethylpolysiloxane, methylphenylpolysiloxane, silicone resins, polyether modified silicone, amino modified silicone, and the like can be used.

For a powder, any powder which is commonly blended into compounded compositions for cosmetic or aesthetic use can be blended into the present invention. Examples are: pearl pigments such as talc, muscovite, synthetic mica, phlogopite, lepidolite, black mica, lithia mica, sericite, vermiculite, kaolin, titanium dioxide, titanium oxide-coated mica, titanium oxide-coated talc, titanium oxide-coated oxybismuth chloride, fish scale flakes and colored titanium oxide-coated mica; metal powder pigments such as aluminum powder and copper powder; inorganic red pigments such as iron oxide (red iron oxide) and iron titanate; inorganic brown pigments such as gamma-iron oxide; inorganic yellow pigments such as yellow iron oxide and loess; inorganic black pigments such as black iron oxide and carbon black; inorganic purple pigments such as mango violet and cobalt violet; inorganic green pigments such as chromium oxide, chromium hydroxide and cobalt titanate; inorganic blue pigments such as ultramarine blue and Berlin blue; zinc white, bentonite, barium sulfate, rectal soap, silious earth, aluminum silicate, calcium silicate, barium silicate, magnesium silicate, stronthium silicate, rectal tungstates, calcium carbonate, magnesium carbonate, chromium oxide, chromium hydroxide, carbon black, alumina, hydroxyapatite, boron nitride, silica, nylon powder, silicone powder, zeolite, benzoguanamine powder, ethylene tetrafluoride powder, polyamide powder, polyester powder, polyethylene powder, polypropylene powder, polystyrene powder, cellulose powder; organic pigments such as red 201, red 202, red 204, red 205, red 220, red 226, red 228, red 405, orange 203, orange 204, yellow 205, yellow 401 and blue 404; organic pigments such as zirconium, barium or aluminum lakes of red 3. red 104, red 106, red 227, red 230, red 401, red 505, orange 205, yellow 4, yellow 5, yellow 202, yellow 203, green 3 and blue 1; and natural colors such as chlorophyll and beta-carotene.

Examples of the ultraviolet light absorbent are: the benzoic acid type such as para-amino benzoate (hereafter abbreviated as “PABA”), glyceryl PABA, n-dimethyl PABA butylether and octyldimethyl PABA; the salicylic acid type such as amino salicylate; and the cinnamic acid type such as octyl cinnamate, ethyl-2,4-diisopropyl cinnamate and octylmethoxy cinnamate; the benzophenone type such as 2,4-dihydroxybenzophenone and 2-hydroxy-4-methoxybenzophenone.

It is also possible to apply an emulsification technique by using purified water, water soluble components and appropriate surface active agents to make an oil-in-water type or water-in-oil type emulsified composition such that the water repellency is not lost.

Bases that can be used in compounded compositions for cosmetic or aesthetic use as an example of the skin preparations of the present invention may be selected from a class of bases that are commonly incorporated as cosmetic components and include the following: animal and plant derived materials such as almond oil, avocado oil, olive oil, turtle oil, bees wax, squalene, lanolin and castor oil; mineral materials such as liquid paraffin, ceresin, paraffin wax, microcrystalline wax, vaseline, candelilla wax, carnauba wax and Japan wax; fatty acids such as palmitic acid and stearic acid; alcohols such as ethanol; higher alcohols such as cetanol, stearyl alcohol, lanolin alcohol, octyl dodecanol and polyvinyl alcohol; esters such as isopropyl myristate, butyl stearate, acetoglyceride, acetylated lanolin, glycerin monostearate, polyoxyethylene monoleate ester, polyethylene glycol monostearic acid ester, and ethyl monooleate; amines such as triethanolamine; ethers such as polyoxyethylene cetyl ether; and silicone oils.

Besides these bases, the following components can be used as required: surfactants such as Spans, Tweens, sugar esters and polyhydric alcoholic esters; mucilages such as acacia gum, tragacanth gum, karaya gum, quince gum, pectin, methyl cellulose, hydroxymethyl propyl cellulose and carboxymethyl cellulose sodium; humectants such as glycerin, propylene glycol, sorbitol, diethylene glycol monoethyl ether and citric acid; extracts from various animals and plants; vitamins; amino acids; activators such as agents to enhance blood circulation; various enzymes; antiinflammatories; saccharides; pigments; biocides or preservatives; antioxidants; and perfumes.

The pigments which are employed to provide the coloring to the compounded compositions for cosmetic or aesthetic use are normally provided as dispersions in castor oil. The pigment and castor oil are preferably present in the lip cosmetic of the invention in an aggregate amount of between about one percent and about ten percent. Pigments of this type are standardized in the cosmetic industry and are identified in that industry by FD&C designations, D&C designations, and natural color designations that are compatible with alcohol solutions. The preferred pigments that are employed include D&C Red No. 6, Barium Lake, D&C Red No. 7 Calcium Lake, D&C Orange No. 5, D&C Red No. 27, FD&C Yellow No. 5, FD&C Blue No. 1, Soloron Silver, Timiron Super Violet, and others. The D&C lake colors are all made with aluminum, calcium, barium, potassium, strontium, or zirconium.

Other pigments which may be employed include those of dye or coal tar origin and chemical compounds used as pigments. Inorganic colors such as iron oxides may be utilized. White pigments may be formed of titanium dioxide, zinc oxide, mica, or pearls. Pigments formed of nitro dyes may be utilized if desired. These dyes contain one atom of nitrogen and two of oxygen. However, only a few nitro dyes are certified by the FD&C or D&C because nitro dyes can be absorbed through the skin, and some are toxic. However, D&C Yellow No. 5 is one acceptable nitro dye which can be used as a component of the pigment of the invention.

Azo pigments may also be utilized. These pigments are characterized by the presence of the azo bond, and monoazo pigments include the greatest number of pigments within this group.

Another group of suitable pigments is the triphenylmethane group. FD&C Blue No. 1 is the most popular dye of the group and is widely used in the cosmetic industry. Xanthene pigments may also be utilized. This group of pigments includes Berry brilliant, which is widely used in lipstick colors, as well as D&C orange.

Certain of the quinoline dyes are also suitable for use as pigments. There are only two certified cosmetic colors in this category, namely D&C Yellow Nos. 10 and 11. These are bright greenish yellow colors.

The anthraquinone dyes are also suitable for use in formulating the pigment required by the compounded compositions for cosmetic or aesthetic use of the invention. These dyes are widely used in cosmetics because they are not affected by light. D&C Violet No. 2 is one example of such a dye suitable for use in the invention.

Indigo dyes are also suitable for use and have been used in compounded compositions for cosmetic or aesthetic use for many years. D&C Blue No. 6 is one example of a suitable indigo dye.

Pigments of vegetable, animal, or mineral origin may also be utilized according to the invention. One suitable pigment of animal origin is cochineal extract. Natural colors and vegetable compound pigments which may be utilized include alkanet, annatto, carotene, chlorophyll saffron and tumeric, beet juice powder, carmine, alkanet root, caramel, grape skin extract, beta carotene, and henna. Hydroxyascetone and indelible dyes may also be utilized in formulating the pigment in the compounded compositions for cosmetic or aesthetic use of the invention.

A preferred embodiment of the compounded compositions for cosmetic or aesthetic use of the invention employ pigments of coloring dispersed in castor oil. This dispersion is present to the extent of between one percent and ten percent of the formulation. The degree of concentration of the pigments in castor oil determines the shade of the coloring. Normally a concentration of between seven and nine percent pigments in castor oil, in the aggregate, is employed in formulating the lip cosmetic. The castor oil acts like a plasticizer and also makes the film formed more flexible.

The compounded compositions of the invention may also employ humectants, such as glycerin or propylene glycol, to keep the lip coating more moist. Flavorings, such as methyl sucrose, and fragrances, absolutes, extracts, and aromatics may also be included in the formulation. In addition, certain chemicals useful as processing aids may be incorporated into the finished product. Titanium dioxide may be used not only to add a white component of color, but also to serve as a sunscreen.

The compounded compositions of the invention may also employ vitamins, minerals, antioxidants, drugs, organic compounds, herbs, proteins, currant extracts, root extracts, enzymes, sorbitol, pectin, and PCA to take advantage of their known coloring, flavoring, moistening, and adhering properties.

The composition according to the invention thus comprises at least one lipophilic component, which may be chosen, for example, from the usual constituents of a fatty phase such as waxes, oils, gums and/or fatty substances which are pasty, hydrocarbon-containing and/or silicone-containing, and possibly volatile, alone or as a mixture.

Other beneficial agents known to one skilled in the art may likewise optionally be included in the composition. Aloe extracts and natural organic acids are exemplary of other beneficial agents. Natural organic acids including alpha-hydroxy acids may act as exfoliants, for example. Lactic acid is an exemplary alpha-hydroxy acid. Preferably any other beneficial agents are derived from botanical sources.

A composition of the invention may be prepared as a liquid mixture and dispensed as a solution or suspension containing the active and inactive ingredients. Another alternative is to prepare the composition of the invention as a molten liquid mixture that can be poured into containers that form the stick shape as the molten mixture cools to a semi-solid state. As overheating may be detrimental to some butters, the temperature of the molten mixture is reduced and/or adjusted to not exceed 160° F. prior to the addition of the butters. Preferably the temperature of the mixture is held in the range of 140° F.-160° F. during mixing steps subsequent to the addition of the butters. Additional mixing steps may include, for example, addition of antioxidants and/or flavoring agents (flavorants).

Upon combination of all ingredients, the molten composition is placed in a stick forming container or containers and allowed to solidify. Optionally, once placed in the container, the forming stick may be subjected to a one or more heating and cooling cycles as it solidifies to optimize formation of the stick. Optionally, the stick-forming container may be a dispensing tube.

Alternatively, the molten composition may be filled into a mold and allowed to solidify in the mold to form a stick. Once formed, the stick is removed from the mold and placed in a dispensing container. Typically, suitable dispensing containers comprise an elevator portion which permits extending the stick from the dispensing container to facilitate application to the user's lips and retraction of the stick back into the container for storage.

The method of the subject invention comprises administering a PDE inhibitor to the outer surface of the lip or lips to enhance or augment the aesthetic appearance of the lip or lips. Specifically, an effective amount of the PDE inhibitor can be applied to the lip or lips to enhance the color (e.g., redness), size, and shape or general fullness of the lip or lips as desired. An effective amount is contained in a composition in accordance with the subject invention, which is dabbed onto the lip or lips to cover and be evenly spread over the lip or lip area. Generally, the application of the composition can be achieved by placing a about one to 10 milliliters of the composition onto the surface of the lip or lips with a finger, or from a stick balm, and allowing the composition comprising the PDE inhibitor to absorb into the lip tissue. The absorption can occur by transport of the composition components into the tissue.

It will be appreciated that the compounds of the subject invention may be used and administered as the raw compound, itself, or as a physiologically acceptable salt or solvate thereof, or in a composition containing the compound, salt or derivative, isomer, or solvate thereof.

Compounds and compositions of the invention may be administered by any suitable route, for example by oral, buccal, sub-lingual, rectal, vaginal, nasal, topical, transdermal, or parenteral (including intravenous, intramuscular, subcutaneous and intracoronary) administration. Topical or transdermal administration is generally preferred.

### Example 1

Sildenafil Lip Formula I

To make 100 g of cream (approximately 3.3 oz) comprising Sildenafil Citrate, 1%; Aminophylline USP Anhydrous, 6%; and Arginine, HCl 12%:
1. Using a paper towel and isopropyl rubbing alcohol, remove the outer blue coating of the Viagra tablets.
2. Using a mortar and pestle, measure 1 g of the Viagra (Sildenafil Citrate) and grind to a fine powder. If needed, sieve powder to remove any remaining tablet coating.
3. Triturate ground Sildenafil Citrate powder, 12 g Arginine Hydrochloride and 6 g Aminophylline USP Anhydrous together in a mortar and pestle.
4. Wet the Step 3 powder with enough Glycerin (approximately 15-30 mL) to form a smooth paste.
5. Using the principles of Geometric Dilution, levigate paste from Step 4 into Humco HRT Botanical base (HUMCO, Texarkana, Tex.), bringing to the final weight of 100 g.
6. Mix until uniform using, preferably, an electronic mortar and pestle.

### Example 2

### Sildenafil Lip Formula II

To make 100 g of cream (approximately 3.3 oz) comprising Sildenafil Citrate, 1%; Naltrexone, 6%; and Arginine, HCl 12%:
1. Using a paper towel and isopropyl rubbing alcohol, remove the outer blue coating of the Viagra tablets.
2. Using a mortar and pestle, measure 1 g of the Viagra (Sildenafil Citrate) and grind to a fine powder. If needed, sieve powder to remove any remaining tablet coating.
3. Triturate Sildenafil Citrate powder of step 2, about 12 g Arginine HCl, and about 6 g Naltrexone HCl together in a mortar and pestle.
4. Wet the powder from Step 3 with enough Glycerin (approximately 15-30 ml) to form a smooth paste.
5. Using the Principles of Geometric Dilution, levigate the paste from Step 4 into a cream base, e.g., HUMCO HRT Botanical Base (HUMCO, Texarkana, Tex.) bringing to the final weight. Mix until uniform.

In order to optimize the shelf-life for the product(s) described herein, it is preferred to protect the formulation from light and from the open air by storing in an opaque and air-tight storage container.

According to USP guidelines, in the absence of stability information, the storage period for compounded Water-Containing Formulations is about 14 days at temperatures between 2° and 8° C.

Although the foregoing invention has been described in some detail by way of illustrations and examples for purposes of clarity of understanding, it will be obvious that certain changes and modifications may be practiced within the scope of the appended claims. Modifications of the above-described modes of practicing the invention that are obvious to persons of skill in the art are intended to be included within the scope of the following claims.

1. A method for enhancing or augmenting the aesthetic appearance of lip tissue, said method comprising applying an effective amount of a phosphodiesterase inhibitor to the outer surface of the lip tissue.

2. The method of claim 1 wherein the phosphodiesterase inhibitor is selected from the group consisting of a pyrazolopyrimidinone, a beta-carboline, an imidazotriazinones, and a methylated xanthine, or a derivative, isomer, hydrate, or salt thereof.

3. The method of claim 1 wherein the phosphodiesterase inhibitor is a pyrazolopyrimidinone.

4. The method of claim 3 wherein the pyrazolopyrimidinone is sildenafil.

5. The method of claim 1 wherein the phosphodiesterase inhibitor is provided in a composition comprising a pyrazolopyrimidinone and a methylated xanthine in a pharmaceutically or cosmetically acceptable carrier.

6. The method of claim 5 wherein the pyrazolopyrimidinone is sildenafil.

7. The method of claim 6 wherein the sildenafil is sildenafil citrate provided in an amount between about 0.5% and about 10% wt/wt of the composition.

8. The method of claim 7 wherein the sildenafil citrate is provided in an amount of about 1% wt/wt of the composition.

9. The method of claim 5 wherein the methylated xanthine is aminophylline.

10. The method of claim 9 wherein the aminophylline is aminophylline anhydrous, provided in an amount between about 1% and about 10% wt/wt of the composition.

11. The method of claim 5 wherein the composition further comprises an amino acid.

12. The method of claim 11 wherein the amino acid is L-arginine in an amount between about 5% to about 15% wt/wt of the composition.

13. The method of claim 1 wherein the method comprises administration of a composition comprising about 1% sildenafil, about 6% aminophylline anhydrous, and about 12% L-arginine and a pharmaceutically or cosmetically acceptable carrier.

14. A composition for enhancing or augmenting the aesthetic appearance of lip tissue, said composition comprising an effective amount of a phosphodiesterase inhibitor.

15. The composition of claim 14 wherein the phosphodiesterase inhibitor is selected from the group consisting of a pyrazolopyrimidinone, a beta-carboline, an imidazotriazinones, and a methylated xanthine, or a derivative, isomer, hydrate, or salt thereof.

16. The composition of claim 14 wherein the phosphodiesterase inhibitor is sildenafil.

17. The composition of claim 16 wherein the sildenafil is sildenafil citrate provided in an amount between about 0.5% and about 10% wt/wt of the composition.

18. The composition of claim 17 wherein the sildenafil citrate is provided in an amount of about 1% wt/wt of the composition.

19. The composition of claim 14 wherein the phosphodiesterase inhibitor is aminophylline.

20. The composition of claim 19 wherein the aminophylline is aminophylline anhydrous, provided in an amount between about 1% and about 10% wt/wt of the composition.

21. The composition of claim 14 wherein the composition further comprises an amino acid.

22. The composition of claim 30 wherein the amino acid is L-arginine in an amount between about 5% to about 15% wt/wt of the composition.

23. The composition of claim 21 wherein the composition comprises about 1% sildenafil, about 6% aminophylline anhydrous, and about 12% L-arginine and a pharmaceutically or cosmetically acceptable carrier.
